(19) United States
(12) Patent Application Publication
(10) Pub. No.: US 2025/0320323 A1
(43) Pub. Date: Oct. 16, 2025

(54) POLYMERIC COMPOSITION

(71) Applicant: Chase Corporation, Westwood, MA (US)

(72) Inventors: Jian Yuan, Sugar Land, TX (US); Mindy Nguyen, Houston, TX (US); Hakim Hazaimeh, Richmond, TX (US); Melanie Urdiales, Houston, TX (US)

(21) Appl. No.: 19/177,265

(22) Filed: Apr. 11, 2025

Related U.S. Application Data
(60) Provisional application No. 63/633,354, filed on Apr. 12, 2024.

(51) Int. Cl. C08F 289/00; A61K 8/91; A61K 8/92; A61Q 19/00; C11C 5/00

(52) U.S. Cl. CPC C08F 289/00; A61K 8/91; A61K 8/922; A61Q 19/00; C11C 5/002

(57) ABSTRACT

This disclosure generally relates to compositions suitable to provide biodegradable polymers, and methods of preparing the polymers thereof. The compositions comprise an alpha-olefin, natural wax or oil (e.g., soybean wax), and an initiator. The polymers can be used for inks, coatings, candle waxes, lubricating oils, personal care compositions, plastics, and adhesives.

### RELATED APPLICATIONS

This application claims priority to Provisional Application Ser. No. 63/633,354, filed Apr. 12, 2024. The entire contents of which are hereby incorporated by reference in its entirety.

### TECHNICAL FIELD

This disclosure generally relates to compositions that are suitable to provide biodegradable polymers comprising an alpha-olefin and soybean wax, and uses of the compositions thereof.

### BACKGROUND

Polyalphaolefins are widely used in commercial fields. However, these polymers are made from pure hydrocarbons, which are hard to degrade and less compatible with natural materials.

Biobased or partially biobased polymers are commercially desired, for example in the cosmetic, personal care, candle, coatings, and adhesives industries. Biobased polymers can provide environmentally friendly, more sustainable, and more bio-compatible materials compared to conventional polymers.

### SUMMARY

In one aspect, described herein are examples of a composition that can include a polymer resulting from combining: an alpha-olefin, a natural wax or oil, and an initiator. A weight ratio of the alpha-olefin to the natural wax or oil is from about 1:20 to 20:1. The natural wax or oil has an iodine value of about 5 to about 220. In an aspect, the polymer resulting from combining the alpha-olefin, natural wax or oil, and initiator, is biodegradable according to OECD 301F.

In an aspect, the weight ratio of the alpha-olefin to the natural wax or oil is about 5:1 to about 1:5, about 2:1 to about 1:2, about 1:1 to about 1:3, about 1.5:1 to about 1:1.5, or about 1.25:1 to about 1:1.25. In an aspect, the weight ratio of the alpha-olefin to the natural wax or oil is about 1:1.

In an aspect, the polymer decreases in mass by at least about 20% under OECD 301F conditions.

In an aspect, the polymer has a softening point of about 0° C. to about 100° C.

In an aspect, the polymer has a softening point of about 0° C. to about 75° C., about 20° C. to about 60° C., or about 25° C. to about 55° C.

In an aspect, the polymer has a natural origin index of greater than 0.5. In an aspect, the polymer has a natural origin index of at least about 0.51, at least about 0.55, at least about 0.6, or at least about 0.65. In an aspect, the polymer has a natural origin index of about 0.5.

In an aspect, the polymer has a hydrocarbon backbone. In an aspect, the polymer has a branched structure. In an aspect, at least 30%, at least 40%, at least 50%, or at least 60% of the polymer branches comprise an ester group.

In an aspect, the polymer has a viscosity at 99° C. of from about 50 cPs to about 700 cPs.

In an aspect, the polymer has a penetration at 25° C. of from about 3 dmm to about 50 dmm. In an aspect, the polymer has a penetration at 25° C. of at least about 15 dmm.

In an aspect, the polymer has an ASTM color of about 0.5. In an aspect, the polymer has a Gardner color of about 1.0.

In an aspect, the polymer has a degradation temperature of less than about 400° C. as measured by thermal gravimetric analysis (TGA) under nitrogen. In an aspect, the polymer has a degradation temperature of about 250° C. to about 400° C. as measured by TGA under nitrogen.

In an aspect, the polymer is soluble in hydrocarbon solvent. In an aspect, the polymer is soluble in at least one of mineral oil, toluene, and xylene.

In an aspect, the polymer is compatible with at least one of vegetable-based oil, hexanol, and 1,2-dimethoxyethane. In an aspect, the polymer is compatible with at least one of jojoba oil, sesame oil, almond oil, and meadowfoam seed oil.

In an aspect, the alpha-olefin has at least 12 carbon atoms. In an aspect, the alpha-olefin comprises C₁₂ to C₅₄ alpha olefins. In an aspect, the alpha-olefin comprises C₁₂ alpha-olefins. In an aspect, the alpha-olefin comprises C₂₄ to C₂₈ alpha-olefins. In an aspect, the alpha-olefin comprises C₃₀ or greater alpha-olefins.

In an aspect, the alpha-olefin is present in the composition in an amount of about 25 wt. % to about 75 wt. %, about 35 wt. % to about 65 wt. %, about 40 wt. % to about 60 wt. %, about 40 wt. % to about 50 wt. %, or about 45 wt. % to about 50 wt. %.

In an aspect, the natural wax or oil is a plant-based wax or oil of soybeans, peanuts, canola, palms, linseed, or a combination thereof. In an aspect, the natural wax or oil is soybean wax. In an aspect, the soybean wax has an iodine value of about 40 to about 55.

In an aspect, the natural wax or oil is present in the composition in an amount of about 25 wt. % to about 75 wt. %, about 35 wt. % to about 65 wt. %, about 40 wt. % to about 60 wt. %, about 40 wt. % to about 50 wt. %, or about 42 wt. % to about 48 wt. %.

In an aspect, the initiator is a peroxide initiator. In an aspect, the initiator is di-tert-butyl peroxide.

In an aspect, the initiator is present in the composition in an amount of about 1 wt. % to about 30 wt. %, about 5 wt. % to about 15 wt. %, or about 5 wt. % to about 10 wt. %.

In one aspect, described herein are compositions that include a polymer resulting from combining: about 25 wt. % to about 75 wt. % alpha-olefin; about 25 wt. % to about 75 wt. % soybean wax; and about 1 wt. % to about 30 wt. % initiator.

In one aspect, described herein are compositions that include a polymer resulting from combining: about 40 wt. % to about 60 wt. % alpha-olefin having at least 12 carbon atoms; about 40 wt. % to about 60 wt. % partially hydrogenated soybean wax; and about 5 wt. % to about 15 wt. % peroxide initiator.

In one aspect, described herein are compositions that include a polymer resulting from combining: an alpha-olefin, soybean wax, and an initiator wherein: the polymer is biodegradable according to OECD 301F; the polymer has a softening point of about 0° C. to about 100° C.; and the polymer has a biobased content of at least 25%.

In one aspect, described herein are compositions that include a polymer resulting from combining: an alpha-olefin, soybean wax, and an initiator wherein: the polymer is biodegradable according to OECD 301F; the polymer has a softening point of about 0° C. to about 75° C.; the polymer has a biobased content of at least 25%; the polymer has a penetration at 25° C. of at least about 15 dmm; and the polymer has a degradation temperature of less than about 350° C. as measured by thermal gravimetric analysis (TGA).

In one aspect, a method of preparing a polymer is described. The method includes the following steps: (a) preparing a composition including an alpha-olefin, natural wax or oil having an iodine value of about 5 to about 220, and an initiator; and (b) heating the composition to a temperature of from about 100° C. to about 200° C. to provide the polymer. In an aspect, the natural wax or oil is soybean wax.

In an aspect, providing the composition comprises mixing the alpha-olefin and the soybean wax to provide a first mixture, and adding the initiator to the first mixture to provide the composition. In an aspect, the initiator is added in multiple portions.

In an aspect, the composition is heated to a temperature of from 150° C. to about 170° C.

In an aspect, the polymer is suitable for use in ink, toner, coating, candle wax, lubricating oil, a personal care composition, a plastic resin, a polylactic acid (PLA) composition, a polishing composition, or a hot melt adhesive.

In an aspect, the polymer is suitable for use in a candle wax. In an aspect, the candle wax comprises from about 0.25 wt. % to about 50 wt. % of the polymer. In an aspect, the polymer is suitable for use in a biobased candle wax. In an aspect, the biobased candle wax comprises soy-based wax, palm wax, coconut wax, beeswax, or a combination thereof. In an aspect, the candle wax has a reduced fat bloom compared to the candle wax without the polymer.

In an aspect, the personal care composition is a cosmetic, a bath product, a lotion, or a fragrance composition. In an aspect, the cosmetic is a color cosmetic or a lipstick. In an aspect, the lotion is a skin care lotion, cleansing balm, or a sunscreen.

Any two or more of the features described in this specification, including in this summary section, can be combined to form implementations not specifically described herein. The details of one or more implementations are set forth in the accompanying drawings and the description below. Other features and advantages will be apparent from the description and drawings, and from the claims.

### DESCRIPTION OF THE DRAWINGS

FIG. 1 depicts an example structure of a polymer prepared from the compositions disclosed herein.

FIG. 2 shows a 13C NMR spectrum of the polymer prepared in Example 1.

FIG. 3 shows a graph of biodegradation (% mass) under OECD 301 conditions for the polymer prepared in Example 1, compared to sodium benzoate and a comparative hydrocarbon polyalphaolefin wax.

FIG. 4A shows a top view of a soy wax without additive, with a hydrocarbon polyalphaolefin additive, and with the polymer additive prepared in Example 1.

FIG. 4B shows a bottom view of a soy wax without additive, with a hydrocarbon polyalphaolefin additive, and with a polymer additive prepared in Example 1.

FIG. 5 shows a temperature/viscosity curve for the polymer prepared in Example 1, a comparative hydrocarbon polyalphaolefin wax, soybean wax, and paraffin wax.

FIG. 6A shows a DSC graph for the polymer prepared in Example 1.

FIG. 6B shows a DSC graph for a comparative hydrocarbon polyalphaolefin.

FIG. 7A shows a TGA graph for the polymer prepared in Example 1.

FIG. 7B shows a TGA graph for a comparative hydrocarbon polyalphaolefin.

### DETAILED DESCRIPTION

The present disclosure describes compositions suitable to provide biodegradable polymers. Such polymers can be useful for a variety of applications, for example, inks, toners, coatings, candle wax, lubricating oil, personal care compositions, plastics, or hot melt adhesives.

Polyalphaolefins are widely used in commercial fields; however, these polymers are made from pure hydrocarbons. Hydrocarbon polyalphaolefins are difficult to degrade and have poor compatibility with natural materials, such as natural waxes.

The polymers disclosed herein can provide a more environmentally friendly, sustainable, and bio-compatible material compared to hydrocarbon polyalphaolefin materials. In some implementations, the polymers disclosed herein are partially biobased polymers prepared from the copolymerization of alpha-olefins with a natural wax (e.g., soybean wax). The compositions disclosed herein can provide polymers with a range of melt points and biobased contents.

The polymers disclosed herein can provide improved biodegradability, recyclability, and sustainability compared to hydrocarbon polyalphaolefin materials. In some implementations, the compositions disclosed herein utilize soybeans as a sustainable raw materials supply. Further, the polymers disclosed herein can provide improved compatibility with natural materials compared to hydrocarbon polyalphaolefins.

The compositions of the present disclosure can comprise a polymer resulting from combining an alpha-olefin, a natural ingredient (e.g., a natural wax or oil), and an initiator. As used herein, the term “polymer” refers to a molecule containing at least two repeat units and includes oligomers. The polymer may have any polymer structure, including but not limited to a linear structure, a branched structure, a hyperbranched structure, or a crosslinked structure. In some implementations, the polymer prepared from the composition has a molecular weight Mn in the range of about 500 to about 10,000 Daltons, or about 1,500 to about 6,000 Daltons, as measured by high temperature GPC in tetrahydrofuran (mobile phase) with polymethyl methacrylate as the standard. In some implementations, the polymer has a polydispersity index (PDI) of from about 1 to about 25.

In some implementations, the alpha-olefin has at least 12 carbon atoms. In some implementations, the alpha-olefin comprises C₁₂ to C₅₄ alpha olefins. In some implementations, the alpha-olefin comprises C₁₂ alpha-olefins. In some implementations, the alpha-olefin comprises C₂₄ to C₂₈ alpha-olefins. In some implementations, the alpha-olefin comprises C₃₀ or greater alpha-olefins. In some implementations, the alpha-olefin comprises at least one of AlphaPlus® C30⁺, AlphaPlus® C₂₄₋₂₈, and 1-dodecene from Chevron Phillips Chemical.

In some implementations, the alpha-olefin is present in the composition in an amount of about 25 wt. % to about 75 wt. %, about 35 wt. % to about 65 wt. %, about 40 wt. % to about 60 wt. %, about 40 wt. % to about 50 wt. %, or about 45 wt. % to about 50 wt. %. In some implementations, the alpha-olefin is present in the composition in an amount of about 25 wt. % to about 50 wt. %. In some implementations, the alpha-olefin is present in the composition in an amount of about 20 wt. % to about 30 wt. %. In some implementations, the alpha-olefin is present in the composition in an amount of about 40 wt. % to about 50 wt. %.

As used herein, a “natural ingredient” refers to an ingredient that falls within the definition of natural ingredients or derived natural ingredients according to ISO 16128-1 (1^{st} edition, 2016 Feb. 15, see Sections 2-3, pgs. 1-3), which is incorporated by reference herein in its entirety. A natural ingredient is obtained only from plants, animals, microorganisms, or minerals. In some implementations, the natural wax or oil is obtained from plants or animals. A derived natural ingredient has greater than 50% natural origin by molecular weight or renewable carbon content.

In some implementations, the natural ingredient is a natural wax or oil. In some implementations, the natural wax or oil is a plant-based wax or oil. In some implementations, the natural wax or oil is a plant-based wax or oil of soybeans, peanuts, canola, palms, linseed, or a combination thereof. In some implementations, the natural wax or oil is an animal wax or oil. In some implementations, the natural wax or oil is fish oil.

In some implementations, the natural wax or oil is partially hydrogenated. In some implementations, the natural wax or oil has an iodine value of about 5 to about 220. In some implementations, the natural wax or oil has an iodine value of about 10 to about 200, about 20 to about 150, about 30 to about 100, about 35 to about 75, or about 40 to about 55.

In some implementations, the natural wax or oil is soybean wax. In some implementations, the soybean wax is partially hydrogenated. In some implementations, the soybean wax is prepared from the hydrogenation of soybean oil and has an iodine value of about 10 to about 80. In some implementations, the soybean wax has an iodine value of about 15 to about 75, about 20 to about 70, about 30 to about 65, about 35 to about 60, or about 40 to about 55. In some implementations, the soybean wax has an iodine value of about 40 to about 55. In some implementations, the soybean wax is Cargill™ ST-130, Cargill™ S-113, Cargill™ S-130, or soybean wax from Archer-Daniels-Midland Company (ADM, Catalog Nos. 885810 or 885820, also named as “hydrogenated soybean oil”).

In some implementations, the natural ingredient (e.g., natural wax or oil) is present in the composition in an amount of about 25 wt. % to about 75 wt. %, about 35 wt. % to about 65 wt. %, about 40 wt. % to about 60 wt. %, about 40 wt. % to about 50 wt. %, or about 42 wt. % to about 48 wt. %. In some implementations, the natural ingredient is present in the composition from about 40 wt. % to about 70 wt. %. In some implementations, the natural ingredient is present in the composition from about 60 wt. % to about 70 wt. %. In some implementations, the natural ingredient is present in the composition from about 40 wt. % to about 50 wt. %.

In some implementations, the composition includes a weight ratio of the alpha-olefin to the natural ingredient (e.g., natural wax or oil) of from about 1:20 to about 20:1. In some implementations, the weight ratio of the alpha-olefin to the natural ingredient is about 5:1 to about 1:5, about 2:1 to about 1:2, about 1:1 to about 1:3, about 1.5:1 to about 1:1.5, or about 1.25:1 to about 1:1.25. In some implementations, the weight ratio of the alpha-olefin to the natural ingredient is about 1:1 to about 1:3. In some implementations, the weight ratio of the alpha-olefin to the natural ingredient is about 3:7. In some implementations, the weight ratio of the alpha-olefin to the natural ingredient is about 1:1.

In some implementations, the initiator is a peroxide initiator. In some implementations, the initiator is di-tert-butyl peroxide. In some implementations, the initiator initiates the polymerization reaction without leaving oxygen atoms in composition (e.g., the resulting polymer product). In some implementations, the composition is substantially free (e.g., 99.5%, 99%, 95%, 90%, 85%, 80%, 75%, 70%, 65%, or 50%) of oxygen atoms from the initiator.

In some implementations, the initiator is present in the composition in an amount of about 1 wt. % to about 30 wt. %, about 5 wt. % to about 15 wt. %, or about 5 wt. % to about 10 wt. %. In some implementations, the initiator is present in the composition in an amount of about 1 wt. % to about 30 wt. %. In some implementations, the initiator is present in the composition in an amount of about 5 wt. % to about 15 wt. %.

The compositions disclosed herein comprise a polymer resulting from combining the alpha-olefin, natural ingredient, and initiator. In some implementations, the polymer is biodegradable according to OECD 301F. As used herein, “biodegradable according to OECD 301F” means that the polymer meets the criteria for either “readily biodegradable” or “inherently biodegradable” as defined by OECD 301F and herein. OECD 301F is a solutions aerobic biodegradation test that determines the biodegradability of a material by measuring oxygen consumption. OECD 301F is described in the OECD Guideline for Testing of Chemicals Adopted Jul. 17, 1992 (see pgs. 48-51), which is incorporated by reference herein in its entirety.

To be considered “readily biodegradable” according to OECD 301F, the pass value is at least 60% biodegradation within a 10-day window. The 10-day window begins when the degree of biodegradation has reached 10% ThOD (theoretical oxygen demand) and must end before day 28 of the test. Compositions which reach the pass levels after the 28-day period are not deemed to be readily biodegradable.

To be considered “inherently biodegradable” according to OECD 301F, the pass value is that the composition must reach at least 20% degradation after the 10-day window (i.e., the polymer reaches at least 20% but less than 60% degradation as measured by OECD 301F). In some implementations, the polymer is “inherently biodegradable” according to OECD 301F.

In some implementations, the polymer decreases in mass by at least about 20% under OECD 301F conditions. In some implementations, the polymer decreases in mass by at least about 20% after 160 days under OECD 301F conditions. In some implementations, the polymer has a faster biodegradation rate than a hydrocarbon polyalphaolefin (i.e., a polymer prepared from a comparable alpha-olefin composition that does not include natural wax or oil).

In some implementations, the polymer is biodegradable according to OECD 301B. OECD 301F is a biodegradation test that determines the biodegradability of a material by measuring CO₂ evolution. OECD 301B is described in the OECD Guideline for Testing of Chemicals Adopted Jul. 17, 1992 (see pgs. 18-22), which is incorporated by reference herein in its entirety. As used herein, “biodegradable according to OECD 301B” means that the polymer meets the criteria for either “readily biodegradable” or “inherently biodegradable” as defined by OECD 301B.

In some implementations, the polymer has a softening point of about 0° C. to about 100° C. In some implementations, the softening point is measured according to ASTM D-36 (e.g., D36/D36M-14, which is incorporated by reference herein in its entirety). ASTM D-36 is a test method for the determination of the softening point of bitumen in the range from 30° C. to 157° C. using a ring-and-ball apparatus dispersed in distilled water 30° C. to 80° C. or USP glycerin (above 80° C. to 157° C.). In some implementations, the polymer has a softening point of about 0° C. to about 75° C., about 20° C. to about 60° C., or about 25° C. to about 55° C. In some implementations, the polymer has a softening point of less than about 75° C.

In some implementations, the polymer has increases hardness and/or softening point as compared to a polymer prepared from natural wax or oil (e.g., soybean wax) without the alpha-olefin.

In some implementations, the polymer has a natural origin index of at least about 0.52, at least about 0.55, at least about 0.6, or at least about 0.65. The natural origin index, as referred to herein, refers to the biobased content of the polymer according to ISO 16128-2 (1st edition, 2017-09, see Section 4.3.2, pg. 3), which is incorporated by reference herein in its entirety. The natural origin index is a value indicating the extent to which a material meets the definition of natural ingredients, derived natural ingredients, or derived mineral ingredients from ISO 16128-1 (1st edition, 2016 Feb. 15, see Sections 2-4, pgs. 1-3), which is incorporated by reference herein in its entirety. The value is calculated as the ratio of the natural origin moiety, as determined by molecular mass, renewable carbon content or any other relevant methods, to the total composition. In some implementations, the natural origin index refers to the ratio of the mass of components in the polymer that are obtained from plants or animals (e.g., the natural wax or oil, and any other natural components) to the total mass of the polymer. For example, a polymer prepared from 51 wt. % natural wax or oil and 49 wt. % alpha-olefin would have a natural origin index of 0.51.

In some implementations, the polymer has a natural origin index of greater than 0.5. In some implementations, the polymer has a natural origin index of between about 0.51 and about 0.9. In some implementations, the polymer has a natural origin index of between about 0.51 and about 0.75. In some implementations, the polymer has a natural origin index of about 0.5. In some implementations, the polymer has a natural origin index of about 0.7.

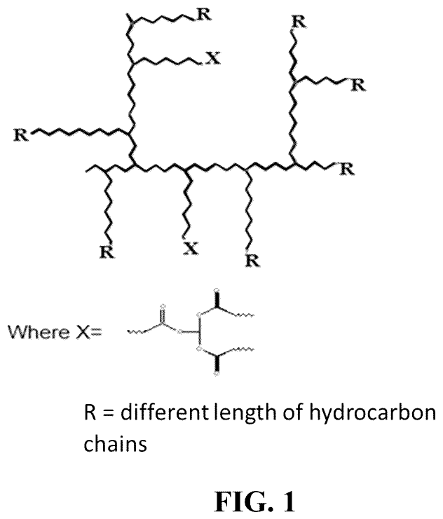

FIG. 1 shows a depiction of an example structure of a polymer prepared from the compositions disclosed herein. In some implementations, the polymer has a hydrocarbon backbone. In some implementations, the polymer is substantially free (e.g., 99.5%, 99%, 95%, 90%, 85%, 80%, 75%, 70%, 65%, or 50%) of epoxides or polyether.

In some implementations, the polymer has a branched structure. In some implementations, at least 30%, at least 40%, at least 50%, or at least 60% of the polymer branches comprise an ester group. In some implementations, at least 60% of the polymer branches comprise an ester group, as measured by 13C NMR using the ratio of the ester peaks to the methyl peaks.

In some implementations, the polymer has a viscosity at 99° C. of from about 50 cPs to about 700 cPs. In some implementations, the polymer has a viscosity at 99° C. of from about 50 cPs to about 200 cPs, about 300 cPs to about 600 cPs, about 200 cPs to about 500 cPs, or about 300 cPs to about 400 cPs. In some implementations, the polymer has a viscosity at 99° C. of from about 75 cPs to about 150 cPs. In some implementations, the polymer viscosity is measured according to ASTM D-3236 (e.g., ASTM D3236-88 (Reapproved 2014), which is incorporated by reference herein in its entirety). ASTM D-3236 is a procedure that tests a sample of molten material in a thermally controlled sample chamber. Apparent viscosity is determined under temperature equilibrium conditions using a precision rotating spindle type viscometer. In some implementations, the polymer viscosity is measured using a Brookfield viscometer having spindle 31 at 99° C.

In some implementations, the polymer has a penetration at 25° C. of from about 3 dmm to about 50 dmm. In some implementations, the penetration is measured according to ASTM D-1321 (e.g., ASTM D 1321-02, which is incorporated by reference herein in its entirety). ASTM D-1321 is a method that determines the empirical estimation of the consistency of waxes by measurement of the extent of penetration of a standard needle using a penetrometer. In some implementations, the polymer has a penetration at 25° C. of at least about 15 dmm.

In some implementations, the polymer has an ASTM color of about 0.5. In some implementations, the ASTM color is measured according to ASTM D-1500 (e.g., ASTM D1500-12 (Reapproved 2017), which is incorporated by reference herein in its entirety). ASTM D-1500 is a method that uses a standard light source and a liquid sample placed in a test container to compare with colored glass disks ranging in value from 0.5 to 8.0 using a three-field comparator. Polymer samples are melted in a standard glass sample jar. In some implementations, the polymer has a Gardner color of about 1.0.

In some implementations, the polymer has a degradation temperature of less than about 400° C. as measured by thermal gravimetric analysis (TGA) under nitrogen. In some implementations, the polymer has a degradation temperature of less than about 350° C. as measured by thermal gravimetric analysis (TGA) under nitrogen. In some implementations, the polymer has a degradation temperature of about 250° C. to about 400° C. as measured by thermal gravimetric analysis (TGA) under nitrogen. In some implementations, the polymer has a degradation temperature of about 250° C. to about 350° C. as measured by thermal gravimetric analysis (TGA) under nitrogen.

In some implementations, the polymer is soluble in hydrocarbon solvent. As used herein, “soluble” means that it is liquid and clear at room temperature. In some implementations, the polymer is soluble in at least one of mineral oil, toluene, and xylene.

In some implementations, the polymer is compatible with at least one of vegetable-based oil, hexanol, and 1,2-dimethoxyethane. As used herein, “compatible” means that a single homogenous phase is present. The homogenous phase may be a gel, solid, or soft solid. In some implementations, the polymer is compatible with plant-based oils, seed-based oils, or both. In some implementations, the polymer is compatible with at least one of jojoba oil, sesame oil, almond oil, and meadowfoam seed oil.

In some implementations, the composition comprises a polymer resulting from combining:
about 25 wt. % to about 75 wt. % alpha-olefin;
about 25 wt. % to about 75 wt. % natural wax or oil having an iodine value of about 5 to about 220; and
about 1 wt. % to about 30 wt. % initiator.

In some implementations, the composition comprises a polymer resulting from combining:
about 25 wt. % to about 75 wt. % alpha-olefin;
about 25 wt. % to about 75 wt. % soybean wax; and
about 1 wt. % to about 30 wt. % initiator.

In some implementations, the composition comprises a polymer resulting from combining:
about 40 wt. % to about 60 wt. % alpha-olefin having at least 12 carbon atoms;
about 40 wt. % to about 60 wt. % partially hydrogenated soybean wax; and
about 5 wt. % to about 15 wt. % peroxide initiator.

In some implementations, the composition comprises a polymer resulting from combining: an alpha-olefin, a natural wax or oil having an iodine value of about 5 to about 220, and an initiator wherein:
the polymer is biodegradable according to OECD 301F;
the polymer has a softening point of about 0° C. to about 100° C.; and
the polymer has a biobased content of at least 25%.

In some implementations, the composition comprises a polymer resulting from combining: an alpha-olefin, soybean wax, and an initiator wherein:
the polymer is biodegradable according to OECD 301F;
the polymer has a softening point of about 0° C. to about 100° C.; and
the polymer has a biobased content of at least 25%.

In some implementations, the composition comprises a polymer resulting from combining: an alpha-olefin, soybean wax, and an initiator wherein:
the polymer is biodegradable according to OECD 301F;
the polymer has a softening point of about 0° C. to about 75° C.;
the polymer has a biobased content of at least 25%;
the polymer has a penetration at 25° C. of at least about 15 dmm; and
the polymer has a degradation temperature of less than about 350° C. as measured by thermal gravimetric analysis (TGA).

A polymer can be prepared using the compositions described herein. An example method of preparing a coating is described below, comprising:
(a) preparing a composition comprising an alpha-olefin, a natural wax or oil having an iodine value of about 5 to about 220, and an initiator; and
(b) heating the composition to a temperature of from about 100° C. to about 200° C. to provide the polymer.

In some implementations, providing the composition comprises mixing the alpha-olefin and the natural wax or oil to provide a first mixture, and adding the initiator to the first mixture to provide the composition. In some implementations, the initiator is added in multiple portions.

Heating the composition can allow for polymerization of the components of the compositions to generate the polymer. In some implementations, the composition is heated to a temperature of from about 150° C. to about 170° C.

In some implementations, the natural wax or oil is soybean wax.

In some implementations, the polymer resulting from combining the alpha-olefin, natural wax or oil, and initiator is suitable for use in ink, toner, coating, candle wax, lubricating oil, a personal care composition, a plastic resin, a polylactic acid (PLA) composition, a polishing composition, or a hot melt adhesive.

In some implementations, the polymer is suitable for use in a candle wax. In some implementations, the candle wax comprises from about 0.25 wt. % to about 50 wt. % of the polymer. In some implementations, the candle wax comprises from about 0.25 wt. % to about 5 wt. % of the polymer.

In some implementations, the polymer is suitable for use in a biobased candle wax. In some implementations, the biobased candle wax comprises soy-based wax, palm wax, coconut wax, beeswax, or a combination thereof. In some implementations, the biobased candle wax comprises soy-based wax. In some implementations, the polymer is suitable for use in a vegetable-based candle wax.

Some candle waxes, such as soy-based candle wax, can have a white frosty appearance which is known as fat blooming from the unsaturated fats and oils. In some implementations, the candle wax has a reduced fat bloom compared to the candle wax without the polymer. In some implementations, the polymer provides improved properties when used in candle wax as compared to hydrocarbon polymers such as polyalphaolefins.

In some implementations, the polymer is used as a candle additive or paraffin-based additive to disperse dye (i.e., uniform dye), increase opacity, reduce surface defects and fat bloom, allow higher fragrance loadings, eliminate oil bleed, provide faster cooling, or a combination thereof. In some implementations, the polymer is suitable for use in soy-based candle wax and can eliminate surface defects, increase adhesion to glass, reduce fat bloom, prevent oil bleed, or a combination thereof. The polymer can be suitable for use and provide benefits to all types of candles, including but not limited to pillars, tapers, jars, and candle melts.

In some implementations, the polymer is suitable for use in a personal care composition. In some implementations, the personal care composition is a cosmetic, a bath product, a lotion, or a fragrance composition. In some implementations, the cosmetic is a color cosmetic or a lipstick. For example, the polymer can replace petroleum-based wax in applications such as lipstick, lotion, hair care, and skin care. In some implementations, the lotion is a skin care lotion, cleansing balm, or a sunscreen. For example, the polymer can be suitable for use in compositions for skin conditions, structuring, and SPF boosters. In some implementations, the polymer is suitable for use in shampoos, body washes, and bath products.

In some implementations, the polymer can improve fragrance depositions, substantivity, and conditioning in personal care applications. For example, the polymer can provide longer lasting fragrance in creams, lotions, and solid fragrances.

In some implementations, the polymer is soluble and compatible with a wide range of cosmetic oils from non-polarity to polarity than other natural waxes, which can provide better consistency texture in oil-based formulations.

In some implementations, the polymer is suitable for use to carry fragrances into detergents, fabric softeners, or fabric finishes.

In some implementations, the polymer is suitable for use in industrial applications as an alternative to low-melting point synthetic waxes and paraffin wax.

In some implementations, the polymer is suitable for use in printing inks or coatings. In some implementations, the polymer can provide printer inks or coatings that are environmentally friendly, food grade, provide a more vibrant and vivid color, or a combination thereof.

In some implementations, the polymer is suitable for use in mold release formulations (e.g., sprays, pastes) that can assist in removing products from a mold. For example, the polymer can be used in food contact mold releases or in a general mold release agent formulation. In some implementations, the properties of the polymer allow for consistent migration to the surface during the molding process. In some implementations, the polymer is mixed with a solvent and the mixture is sprayed on a substrate. When the solvent is evaporated, the substrate is coated with a polymer film which can help to demold a non-compatible resin out from the substrate. In some implementations, the polymer is suitable for spray or paste on a mold as a release agent during the molding of a plastic or foam.

In some implementations, the polymer is suitable for use as a processing aid for plastic resins. For example, the polymer can provide better throughput and lower torque when used for processing plastic resins through an extruder.

In some implementations, the polymer is suitable for use as an additive to improve dispersion filler materials in polymer resins.

In some implementations, the polymer is suitable for use as an additive for polylactic acid (PLA).

In some implementations, the polymer is suitable for use in polishing applications. In some implementations, the polymer is compatible with both synthetic and natural waxes, which allow for good polishing properties. For example, the polymer can promote high gloss and provide durability and water resistance.

In some implementations, the polymer is suitable for use in hot melt adhesives. For example, the polymer can promote adhesion with a wide range of substrates, including engineering resins. In some implementations, the polymer can adhere to both non-polar and polar resins.

### EXAMPLES

### Example 1. Polymer Prepared from Soybean Wax and C₃₀₊ Olefin

Soybean wax (150 grams) and C30+ olefin (150 grams) were heated to 160° C. with stirring. DTBP (Di-tert-butyl peroxide, 25 grams) was added in 4 portions over 5 hours. The reaction temperature was maintained at 160° C. for an additional 1 hour. The reaction mixture is purged at 150° C. for one hour under a nitrogen sparge. The polymer product was tested with viscosity measurement: 433 cPs@210F (99° C.).

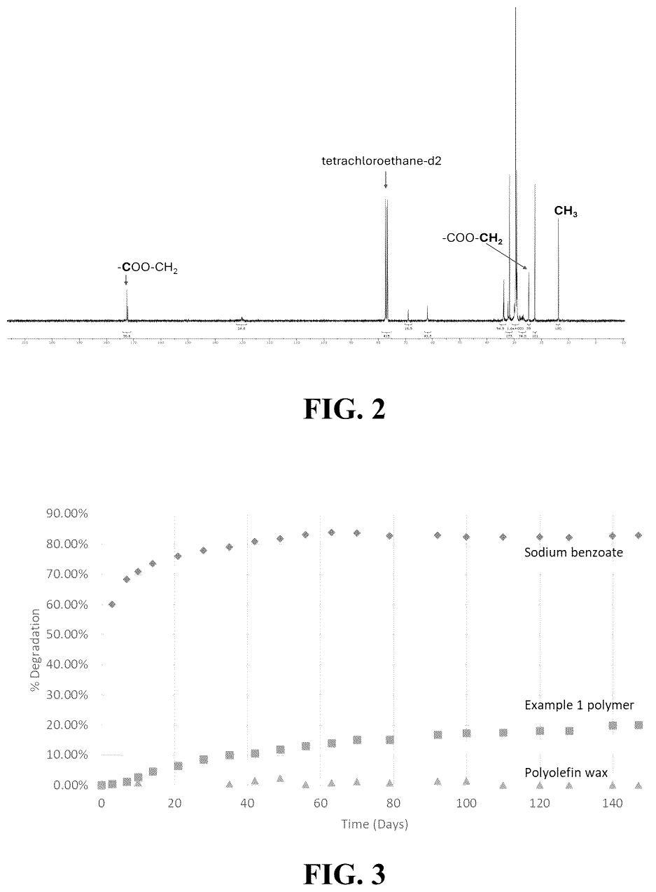

FIG. 2 shows the 13C NMR spectrum of the polymer product in tetrachloroethane-d2. The NMR instrument was a Varian 300 MHz and the NMR temperature was 100° C. The NMR tube diameter was 1 cm. The percent of polymer branches having an ester peak was calculated to be 57% by comparing the ratio of the —COO—CH₂ peak to the CH₃ peak.

### Example 2. Polymer Prepared from Soybean Wax and C₂₄₋₂₈ Olefin

Soybean wax (220 grams) and C₂₄₋₂₈ olefin (94 grams) were heated at 160° C. with stirring. DTBP (Di-tert-butyl peroxide, 25 grams) was added in 4 portions over 5 hours. The reaction temperature was maintained at 160° C. for an additional 1 hour. The reaction mixture was purged at 150° C. for one hour under a nitrogen sparge. The polymer product was tested with viscosity measurement: 219 cPs@210F (99° C.).

### Example 3. Polymer Prepared from Soybean Wax and C₁₂ Olefin

Soybean wax (350 grams) and C12 olefin (350 grams) were heated to 160° C. with stirring. DTBP (Di-tert-butyl peroxide, 89 grams) was added in 4 portions over 15 hours. The reaction temperature was maintained at 160° C. for an additional 1 hour. The reaction mixture is purged at 150° C. for one hour under a nitrogen sparge. The polymer product was tested with viscosity measurement: 620 cPs@90F (99° C.).

### Softening Point Measurements

Softening points were determined using ASTM D36/D36M-14 using a ring-and-ball apparatus. Two horizontal disks of bitumen, cast in shouldered brass rings, were heated at a controlled rate in a liquid bath while each supported a steel ball. The softening point is reported as the mean of the temperatures at which the two disks soften enough to allow each ball, enveloped in bitumen, to fall a distance of 25 mm (1.0 inch).

### Penetration Measurements

Penetration was determined using ASTM D 1321-02. The polymer sample was melted, heated to 17° C. above its congealing point, poured into a container, and then air cooled under controlled conditions. The polymer sample was then conditioned in a water bath. Penetration was measured with a penetrometer, which applied a standard needle to the sample for 5 seconds under a load of 100 g.

Physical Properties of Polymers Prepared from Compositions of Examples 1-3

Selected physical properties of the polymers prepared from example compositions disclosed herein are shown in Tables 1 and 2. Table 2 also includes comparative data for hydrocarbon polyalphaolefins. Polyalphaolefin 1, as referred to herein, was a hard polyalphaolefin prepared from a long alpha-olefin. Polyalphaolefin 2, as referred to herein, was a soft polyalphaolefin prepared from a short alpha-olefin.

TABLE 1
Physical properties of polymers

| Example Composition | Example 1 | Example 2 | Example 3 |
|---|---|---|---|
| Alpha-Olefin | C30+ | C24-28 | C12 (liquid) |
| Soybean wax to alpha- | 50.1:49.9 | 7:3 | 50.1:49.9 |
| olefin | | | |
| Viscosity at 210° F. (99° | 433 | 219 | 653 |
| C.)/cPS | | | |
| DSC/° C. | 52.09 | 28.99 | n.d. |
| Penetration at 77° | 18 | 58 | n.d. |
| F./dmm | | | |
| Softening Point/° F. | 150 | 113.5 | n.d. |
| Color/ASTM | 0.5 | 0.5+ | 0.5− (ASTM) |
| | | | 1.0 (Gardner) |
| Flash OC/° F. | >500 | 605 | 510 |

“n.d.” means not determined.
“OC” means open cup.

TABLE 2
Physical properties of example polymers and
comparative hydrocarbon polyalphaolefins

| | Softening | Viscosity | Penetration | Natural |
|---|---|---|---|---|
| Product | Point/° C. | (@99° C.)/cPs | @ 25° C./dmm | Origin Index |
| Polyalphaolefin 1 (fully | 74 | 345 | 5 | 0 |
| hydrocarbon) | | | | |
| Polyalphaolefin 2 (fully | 54 | 360 | 12 | 0 |
| hydrocarbon) | | | | |
| Example 1 polymer | 65 | 433 | 18 | 0.51 |

### Molecular Weight Measurements by Gel Permeation Chromatography (GPC)

The polymer samples were prepared in duplicate as approximately 1 mg/ml solutions using the mobile phase, tetrahydrofuran (THF) as the diluent. The sample solutions were left to sit overnight at ambient temperature, then heated to 50° C. for 1 hour to aid dissolution. Upon visual inspections, all samples appeared to be completely dissolved in solution. The sample preparations were filtered through a 0.45 μm nylon membrane and analyzed using a Waters Alliance Separations Module equipped with a polystyrene/divinylbenzene column set with THE as the mobile phase and refractive index (RI) detection. Narrow PMMA standards ranging from 2.2 million Daltons to 540 Daltons were used for calibration.

GPC analysis showed that the samples had a broad, multimodal molecular weight distribution. Example polymer 1 had a Mn (Daltons) of 5660 and a PDI (Mw/Mn) of 19.5.

### OECD 301F Biodegradation Test Conditions

An example OECD 301F biodegradation test included two blank controls, one reference control, and two test item groups. The blank controls each contained mineral medium and inoculum, and were used to measure the background oxygen consumption by the inoculum alone. The reference control consisted of mineral medium, inoculum, and sodium benzoate. Sodium benzoate is a substance known to be biodegradable, and has been widely used as a reference chemical in ready biodegradation tests. Both the reference and test item were dosed at a concentration of 60 mg ThOD/L.

Determination of ThOD. If the formula or elemental composition is known, for compound C_{c}HₕCl_{cl}NₙNa_{Na}OₒPₚSₛ, the ThOD, without nitrification, would be:

$${ThOD}_{NH 3} = \frac{16 [ 2 c + 1 / 2 ( h - cl - 3 n ) + 3 s + 5 / 2 p + 1 / 2 na - o ] mg / mg}{MW}$$

with nitrification (less common):

$${ThOD}_{NO 3} = \frac{16 [ 2 c + 1 / 2 ( h - cl ) + 5 / 2 n + 3 s + 5 / 2 p + 1 / 2 na - o ] mg / mg}{MW}$$

If the elemental composition is unknown, COD may be determined as an alternative. Details for COD determination can be referred to standard methods such as ISO 15705, ASTM D1252, and HACH Method 8000.

Reference Item. A stock solution of sodium benzoate was be prepared at a nominal concentration of 2,500 mg ThOD/L in test medium. Volumetric addition of the stock solution was then followed. The dosing volume of the reference substance stock solution was calculated based on the nominal value of the stock solution and the target concentration in the working solution. The pH of the stock solution was determined and adjusted to 7.4+0.2 if necessary. The stock solution may be stored in a refrigerator for a maximum of three days.

Mineral medium. The mineral medium is a mixture of various anions and cations, which provides essential elements for the growth of microorganisms and maintains the pH of the system at a constant level during the biodegradation processes. The preparation procedures are as follows.

The following stock solutions were prepared using reagent grade reagents:

| | | |
|---|---|---|
| (a) Potassium dihydrogen orthophosphate, KH₂PO₄ | 8.50 | g |
| Dipotassium hydrogen orthophosphate, K₂HPO₄ | 21.75 | g |
| Disodium hydrogen orthophosphate dihydrate, | 33.40 | g |
| Na₂HPO₄•2H₂O | | |
| Ammonium chloride, NH₄Cl | 0.50 | g |
| Dissolved in water and made up to 1 L. The pH of the | | |
| solution should be 7.4. | | |
| (b) Calcium chloride, anhydrous, CaCl₂ | 27.50 | g |
| or | | |
| Calcium chloride dihydrate, CaCl₂•2H₂O | 36.40 | g |
| Dissolved in water and made up to 1 L. | | |
| (c) Magnesium sulphate heptahydrate, MgSO₄•7H₂O | 22.50 | g |
| Dissolved in water and made up to 1 L. | | |
| (d) Iron (III) chloride hexahydrate, FeCl₃•6H₂O | 0.25 | g |
| Dissolved in water and made up to 1 L. | | |

To prepare the mineral medium, 10 mL of solution (a), and 1 mL of solutions (b), (c) and (d) were mixed with 800 mL water and made up to 1 L with water.

Inoculum. The inoculum was activated sludge collected from the end of the aeration tank of Twinsburg Wastewater Treatment Plant (Twinsburg, OH). The sludge was filtered to remove course particles. After settling for 15 min, the supernatant was then decanted and mineral medium was then added to the concentrated solids to bring the suspended solid to 3-5 g/L. Mineral medium can also help to maintain the pH at around 7.4. The resulting solution was aerated for up to 7 days at room temperature (known as a pre-conditioning process), and then added into working solutions to achieve 30 mg/L of total suspended solids to initiate the biodegradation.

Apparatus and System setup. The test was performed in 500 mL tightly closed bottles, each filled with 333.3 mL of working solution so that the air to solution ratio is 1:2. This ratio is appropriate to provide enough oxygen to maintain an aerobic condition even if the sample is completely degraded, but also ensures a significant oxygen level decrease so that the amount of oxygen consumption can be accurately determined by the instrument.

Preparation of working solutions and bottles. The working solutions were prepared based on the following steps:
(1) Added an appropriate amount of pre-conditioned activated sludge solution into mineral medium in order to achieve 30 mg/L of total suspended solid dosage in the final working solution.
(2) Added an appropriate amount of test item or reference item to achieve 60 mg ThOD/L (did not include this step for blank control group).
(3) Dispensed each prepared solution or suspension immediately into the respective group of reactors at a volume of 333.3 mL per reactor.
(4) Tightly closed the caps to avoid air exchange between the bottles and the outside.

Test initiation. Once the working bottles were prepared, the zero-time oxygen was analyzed immediately. The bottles were placed into an incubator on a magnetic stirrer in the absence of light for incubation at 22±2° C.

Test termination. On the last day of study, once all the oxygen measurements were taken, the incubation was stopped to terminate the tests.

Degradation percentage determination. During the first ten days, the analysis was generally made every three to four days, and then every five to seven days until the end of the test.

Oxygen consumption (mg (2) for each reactor was first obtained (considering oxygen from both the headspace and the solution), and then the BOD was calculated as mg O₂/mg test substance:

$$\begin{matrix}BOD = \frac{mg {O}_{2} uptake by test substance - mg {O}_{2} uptake by blank}{mg test substance in vessel} \\ = mg {O}_{2} / mg substance\end{matrix}$$

The biodegradation percentage is then calculated by:

$$\% degradation = \frac{BOD ( mg {O}_{2} / mg test substance )}{ThOD or COD ( mg {O}_{2} / mg test substance )} \times 100$$

The reference substance must achieve 60% degradation within the 10-day window to be considered biodegradable.

### Example 4. Biodegradation of Polymers Disclosed Herein (OECD 301F Test)

The Example 1 polymer was tested for biodegradability under OECD 301F test, in comparison to sodium benzoate (a substance known to be readily biodegradable) and a hydrocarbon polyalphaolefin wax prepared from a composition that did not include soybean wax (Polyalphaolefin 1). The biodegradation data (% degradation) is shown in Table 3.

TABLE 3
Biodegradation of Example 1 polymer, sodium benzoate,
and a hydrocarbon polyalphaolefin under OECD 301F test.

| | Sodium benzoate | | Example 1 polymer | | Polyalphaolefin 1 | |
|---|---|---|---|---|---|---|
| Time (day) | Mean | Error | Mean | Error | Mean | Error |
| 0 | 0.0% | — | 0.0% | 0.0% | 0.0% | 0.0% |
| 3 | 60.1% | — | 0.5% | 0.3% | −0.3% | 2.4% |
| 7 | 68.2% | — | 1.2% | 0.2% | −0.5% | 1.3% |
| 10 | 70.9% | — | 2.6% | 0.2% | 0.7% | 1.2% |
| 14 | 73.5% | — | 4.6% | 0.7% | −1.9% | 2.5% |
| 21 | 75.9% | — | 6.4% | 0.9% | −0.1% | 2.1% |
| 28 | 77.9% | — | 8.4% | 1.2% | −2.3% | 0.8% |
| 35 | 79.0% | — | 9.9% | 1.2% | 0.4% | 1.5% |
| 42 | 80.8% | — | 10.6% | 0.9% | 1.5% | 2.0% |
| 49 | 81.8% | — | 11.9% | 0.7% | 2.3% | 1.9% |
| 56 | 83.0% | — | 13.0% | 1.0% | 0.3% | 1.8% |
| 63 | 83.9% | — | 13.9% | 0.8% | 0.7% | 1.8% |
| 70 | 83.7% | — | 15.1% | 0.7% | 1.1% | 1.2% |
| 79 | 82.7% | — | 15.0% | 1.1% | 0.8% | 0.5% |
| 92 | 82.9% | — | 16.8% | 0.5% | 1.3% | 0.6% |
| 100 | 82.4% | — | 17.2% | 0.7% | 1.4% | 0.4% |
| 110 | 82.4% | — | 17.4% | 0.8% | — | — |
| 120 | 82.4% | — | 18.0% | 0.5% | — | — |
| 128 | 82.2% | — | 18.1% | 0.7% | — | — |
| 140 | 82.7% | — | 19.9% | 0.5% | — | — |
| 147 | 82.8% | — | 20.1% | 0.8% | — | — |

Example 1 polymer was “Inherently Biodegradable” according to OECD 301 after 147 days, while the hydrocarbon wax was not degradable at the same time. “Inherently Biodegradable” according to OECD 301 means that the composition reaches at least 20% biodegradation after the 10-day window. As shown in Table 4, the Example 1 polymer reached 20.1% biodegradation after 147 days under the OECD 301F test, and therefore is “Inherently Biodegradable” according to OECD 301F.

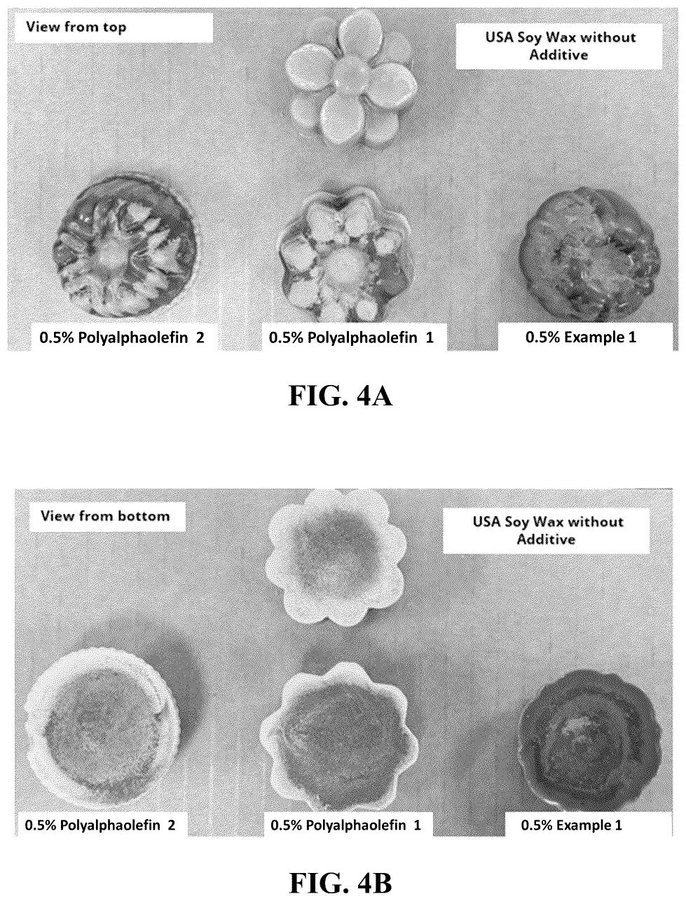

FIG. 3 shows the percent degradation by mass of Example 1 polymer compared to sodium benzoate and hydrocarbon polyalphaolefin wax. The study was conducted in compliance with ISO/IEC 17025:2017, and the OECD and USEPA Good Laboratory Practice Standard.

After 160 days, the Example 1 polymer lost 20% mass. By definition according to OECD 301F, the polymer is an “Inherently Biodegradable” wax.

### Example 5. Solubility of Example 1 Polymer and Hydrocarbon Version

The solubility of the Example 1 polymer is shown in Table 4, as well as a comparative fully hydrocarbon polyalphaolefin (Polyalphaolefin 1). “S” refers to soluble, “C” refers to compatible, “SC” refers to slightly compatible, and “NC” refers to not compatible. The polymer was considered “compatible” if it mixed well in the solvent without any separation, but the solution was not clear. The polymer was considered “slightly compatible” if the polymer partially mixed in the solvent (e.g., over time, some of the polymer was settled in big particle in the solvent).

TABLE 4
Solubility of example polymers

| | Example 1 | Polyalphaolefin 1 |
|---|---|---|
| Water | NC | NC |
| Mineral oil | S | S |
| Vegetable based oil | C | C |
| Butyl alcohol | SC | NC |
| Hexanol | C | NC |
| 1,2-dimethoxyethane | C | NC |
| (monoglyme) | | |
| Di(propylene glycol) | NC | NC |
| methyl ether | | |
| (DPM) | | |
| Toluene | S | S |
| Xylene | S | S |

### Example 6. Candle Wax Including Example Polymers

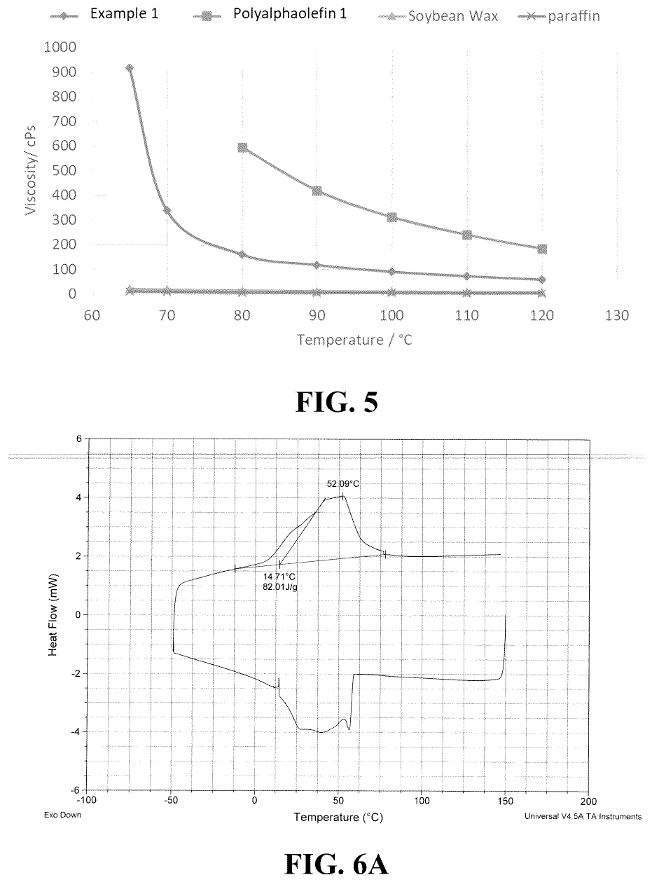

The Example 1 polymer was included in USA soy wax melts in an amount of 0.5 wt. %. USA soy wax melts without the example polymer additive had a severe level of fat bloom. As shown in FIGS. 4A and 4B, the soy wax melts including the example 1 polymer had a reduced fat bloom as compared to fully hydrocarbon polyalphaolefin wax additives (Polyalphaolefin 1 and Polyalphaolefin 2).

### Example 7. Candle Wax Including Example Polymers

The viscosity of the polymers was measured according to ASTMD3236-88 (Reapproved 2014) using a Brookfield viscometer having spindle 31 at varying temperatures.

Table 5 shows the viscosity at various temperatures for the Example 1 polymer, a comparative hydrocarbon polyalphaolefin, soybean wax, and paraffin. The hydrocarbon polyalphaolefin had a higher viscosity at each temperature than the Example 1 polymer.

TABLE 5
Viscosities at varying temperatures

| | Viscosity (cPs) | | | |
|---|---|---|---|---|
| | Example 1 | Hydrocarbon | | |
| Temp/° C. | polymer | polyalphaolefin | Soybean Wax | Paraffin |
| 65 | 917.3 | solid | 20.4 | 9.6 |
| 70 | 337.4 | solid | 17.8 | 8.4 |
| 80 | 160.2 | 593.9 | 14 | 6 |
| 90 | 118.2 | 419.3 | 11.4 | 5.4 |
| 100 | 90.3 | 312.1 | 9.6 | 5.1 |
| 110 | 72.9 | 241.1 | 8.16 | 3.9 |
| 120 | 59.1 | 185.1 | 7.08 | 3.6 |

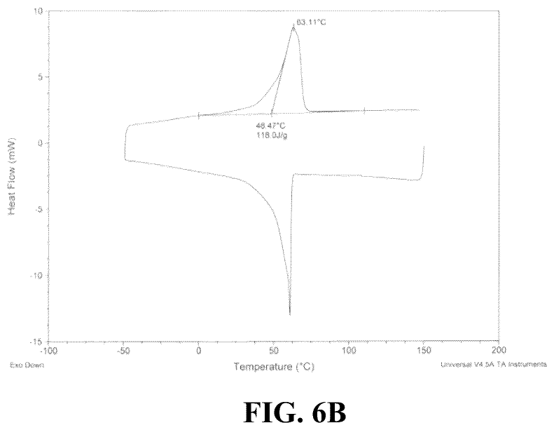

FIG. 5 shows the temperature vs. viscosity curve for the Example 1 polymer, the hydrocarbon polyalphaolefin, soybean wax, and paraffin. Without wishing to be bound by theory, the polymers disclosed herein have natural components which provide more polymer behavior as compared to soybean and paraffin wax, as can be seen in FIG. 5.

### Example 8. DSC of Example Polymers

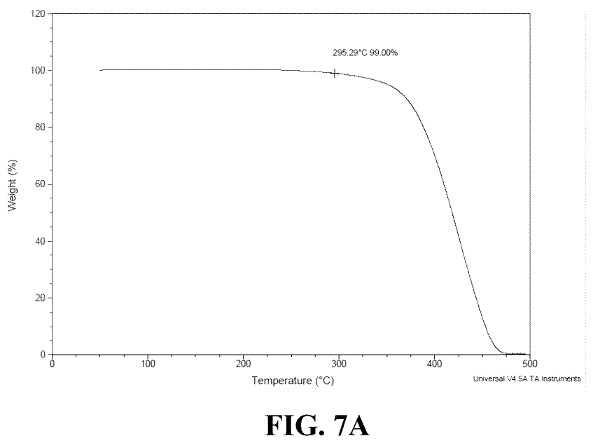

FIG. 6A shows the DSC (Differential Scanning calorimetry) of the Example 1 polymer having 50% biobased content. FIG. 6B shows the DSC for a comparative polyalphaolefin that is fully hydrocarbon. As can be seen in FIGS. 6A and 6B, adding 50% biobased content dropped the peak melting point of the polymer as compared to the fully hydrocarbon polymer.

### Example 9. TGA of Example Polymers

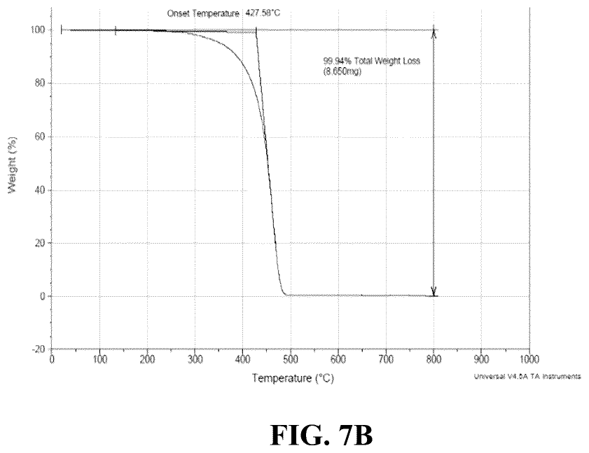

FIG. 7A shows the TGA (Thermal Gravimetric Analysis) of the Example 1 polymer having 50% biobased content. FIG. 7B shows the TGA for a comparative polyalphaolefin that is fully hydrocarbon. As can be seen in FIGS. 7A and 7B, adding 50% biobased content dropped the degradation temperature by about 130° C. The Example 1 polymer begins to degrade at about 295° C. whereas the fully hydrocarbon polymer begins to degrade at about 427° C.

As used herein, and unless otherwise specified, the term “about,” when used in connection with a numeric value or range of values is to indicate that the value or range of values may deviate to an extent deemed reasonable to one of ordinary skill in the art. In general, the term “about” is used herein to modify a numerical value above and below the stated value by a variance of 10% unless otherwise specified. For temperature values in degree Celsius (° C.), the term “about” is used to indicate the stated value±3° C. It is well known that instrument variation and other factors can affect the numerical values. The term “about” is to accommodate these variations. Each reference, including all non-patent documents, patents, and patent applications, cited in the present application is incorporated herein by reference in its entirety.

1. A composition comprising a polymer resulting from combining: an alpha-olefin, a natural wax or oil, and an initiator, wherein:
a weight ratio of the alpha-olefin to the natural wax or oil is from about 1:20 to 20:1;
the natural wax or oil has an iodine value of about 5 to about 220; and
the polymer is biodegradable according to OECD 301F.

2. The composition of claim 1, wherein the weight ratio of the alpha-olefin to the natural wax or oil is about 5:1 to about 1:5, about 2:1 to about 1:2, about 1:1 to about 1:3, about 1.5:1 to about 1:1.5, or about 1.25:1 to about 1:1.25.

3. The composition of claim 1, wherein the weight ratio of the alpha-olefin to the natural wax or oil is about 1:1.

4. The composition of claim 1, wherein the polymer decreases in mass by at least about 20% under OECD 301F conditions.

5. The composition of claim 1, wherein the polymer has a softening point of about 0° C. to about 100° C.

6.-15. (canceled)

16. The composition of claim 1, wherein the polymer has a branched structure; and
wherein at least 30% of the polymer branches comprise an ester group.

17.-19. (canceled)

20. The composition of claim 1, wherein the polymer is soluble in hydrocarbon solvent.

21.-24. (canceled)

25. The composition of claim 1, wherein the alpha-olefin comprises C₁₂ to C₅₄ alpha olefins.

26. The composition of claim 1, wherein the alpha-olefin comprises C₁₂ alpha-olefins.

27. The composition of claim 1, wherein the alpha-olefin comprises C₂₄ to C₂₈ alpha-olefins.

28. (canceled)

29. The composition of claim 1, wherein the alpha-olefin is present in the composition in an amount of about 25 wt. % to about 75 wt. %, about 35 wt. % to about 65 wt. %, about 40 wt. % to about 60 wt. %, about 40 wt. % to about 50 wt. %, or about 45 wt. % to about 50 wt. %.

30. The composition of claim 1, wherein the natural wax or oil is a plant-based wax or oil of soybeans, peanuts, canola, palms, linseed, or a combination thereof.

31. The composition of claim 1, wherein the natural wax or oil is soybean wax.

32. (canceled)

33. The composition of claim 1, wherein the natural wax or oil is present in the composition in an amount of about 25 wt. % to about 75 wt. %; about 35 wt. % to about 65 wt. %, about 40 wt. % to about 60 wt. %, about 40 wt. % to about 50 wt. %, or about 42 wt. % to about 48 wt. %.

34. The composition of claim 1, wherein the initiator is a peroxide initiator.

35. (canceled)

36. The composition of claim 1, wherein the initiator is present in the composition in an amount of about 1 wt. % to about 30 wt. %, about 5 wt. % to about 15 wt. %, or about 5 wt. % to about 10 wt. %.

37. (canceled)

38. A composition comprising a polymer resulting from combining:
about 40 wt. % to about 60 wt. % alpha-olefin having at least 12 carbon atoms;
about 40 wt. % to about 60 wt. % partially hydrogenated soybean wax; and
about 5 wt. % to about 15 wt. % peroxide initiator.

39.-40. (canceled)

41. A method of preparing a polymer, comprising:
(a) preparing a composition comprising an alpha-olefin, natural wax or oil having an iodine value of about 5 to about 220, and an initiator; and
(b) heating the composition to a temperature of from about 100° C. to about 200° C. to provide the polymer.

42.-45. (canceled)

46. The composition of claim 1, wherein the polymer is suitable for use in ink, toner, coating, candle wax, lubricating oil, a personal care composition, a plastic resin, a polylactic acid (PLA) composition, a polishing composition, or a hot melt adhesive.

47. The composition of claim 46 wherein the polymer is suitable for use in a candle wax.

48.-54. (canceled)
